(19) United States
(12) Patent Application Publication
(10) Pub. No.: US 2024/0000754 A1
(43) Pub. Date: Jan. 4, 2024

(54) METHODS OF TREATMENT OF LIVER FAILURE

(71) Applicant: GENFIT, LOOS (FR)

(72) Inventors: Philippe DELATAILLE, Ronchin (FR); Robert WALCZAK, Lille (FR); Corinne FOUCART, La Madeleine (FR); Vanessa LEGRY, Emmerin (FR); Nicolas STANKOVIC VALENTIN, Mouscron (BE); Simon DEBAECKER, Pont-a-Marcq (FR); Rémy HANF, Lille (FR)

(21) Appl. No.: 18/252,760

(22) PCT Filed: Nov. 16, 2021
(86) PCT No.: PCT/EP2021/081876

§ 371 Date: May 12, 2023

(30) Foreign Application Priority Data
Nov. 17, 2020 (EP) 20306396.1
Sep. 28, 2021 (EP) 21199623.6

(51) Int. Cl. A61K 31/426; A61P 1/16; A61P 25/00

(52) U.S. Cl. CPC A61K 31/426; A61P 1/16; A61P 25/00

(57) ABSTRACT

The invention relates to the treatment or prevention of liver failure.

The invention relates to the treatment or prevention of liver failures.

### BACKGROUND OF THE INVENTION

Liver failure is a severe inability of the liver to perform its normal metabolic functions. Manifestations of liver failure include acute liver failure, cirrhosis, and acute on chronic liver failure.

### Acute Liver Failure (ALF)

The term ALF describes a disorder characterized by an acute loss of liver function in the absence of pre-existing chronic liver disease. ALF has also been referred to as fulminant hepatic failure, acute hepatic necrosis, fulminant hepatic necrosis, and fulminant hepatitis.

ALF is a rare and severe consequence of abrupt hepatocyte injury, and can evolve over days or weeks to a lethal outcome. A variety of insults to liver cells result in a consistent pattern of rapid-onset elevation of aminotransferases, altered mentation, and disturbed coagulation. The absence of existing liver disease distinguishes ALF from liver failure due to end-stage chronic liver disease (decompensated cirrhosis and acute-on-chronic liver failure, ACLF). In ALF, substances that lead to hepatocyte injury cause either direct toxic necrosis, or apoptosis and immune injury, which is a slower process. The time from the onset of symptoms to the onset of hepatic encephalopathy distinguishes the different forms of acute liver failure: a direct, very rapid injury (within hours), referred to as hyperacute liver failure; and a slower, immune-based injury (days to weeks), considered acute or subacute. The term “hepatic encephalopathy”, or HE, as used herein refers to the occurrence of confusion, altered level of consciousness and coma as a result of liver failure. In the advanced stages it is called hepatic coma or coma hepaticum.

The five most prevalent causes of ALF in developed countries are paracetamol (acetaminophen) toxicity, ischaemia, drug-induced liver injury, hepatitis B, and autoimmunity, which account for nearly 80% of cases. Hepatitis A, B, and E are the main causes of ALF in developing countries. The remaining causes of ALF comprise fewer than 15% of the total and include heat stroke, pregnancy-associated injury (e.g., acute fatty liver of pregnancy and HELLP [haemolysis, elevated liver enzyme, and low platelet] syndrome), Budd-Chiari syndrome, nonhepatotrophic viral infections such as herpes simplex, and diffusely infiltrating malignancies.

Untreated, the prognosis is poor, so timely recognition and management of patients with acute liver failure is crucial. Whenever possible, patients with acute liver failure should be managed in an intensive care unit at a liver transplantation center.

### Cirrhosis

The term “cirrhosis” as used herein, refers to a condition characterized by replacement of liver tissue by fibrosis and regenerative nodules which lead to loss of liver function up to decompensation. Ascites (fluid retention in the abdominal cavity) is the most common complication associated with cirrhosis decompensation. It is associated with a poor quality of life, increased risk of infection and poor long-term outcome. Other potentially life-threatening complications are hepatic encephalopathy (confusion and coma) and bleeding from esophageal varices. Cirrhosis has many possible manifestations. These signs and symptoms may be either as a direct result of the failure of liver cells or secondary to the resultant portal hypertension. Effects of portal hypertension include splenomegaly, gastroesophageal varices, and portocollateral circulation as a result of formation of venous collateral veins between portal system and the periumbilical veins as a result of portal hypertension.

Cirrhosis is divided in two clinical categories: compensated and decompensated cirrhosis.

The term “compensated cirrhosis” as used herein, means that the liver is heavily scarred but can still perform many important bodily functions. Patients suffering from compensated cirrhosis experience few or no symptoms and can live without serious clinical complications. Patients at early stages of compensated cirrhosis are characterized by low levels of portal hypertension and lack of esophageal varices. Patients at advanced stages of compensated cirrhosis are characterized by higher levels of portal hypertension and presence of esophageal varices but without ascites and without bleeding.

The term “decompensated cirrhosis” as used herein, means that the liver is extensively scarred and unable to function properly. Patients suffering from decompensated cirrhosis develop a variety of symptoms such as fatigue, loss of appetite, jaundice, weight loss, ascites and/or edema, hepatic encephalopathy and/or bleeding. Patients at early stages of decompensated cirrhosis are characterized by the presence of ascites with or without esophageal varices in a patient that has never bled. Patients at advanced stages of decompensated cirrhosis are characterized by more sever ascites alone or in association with bleeding, bacterial infections and/or hepatic encephalopathy. Complications associated with decompensated cirrhosis such as ascites, edema, bleeding problems, bone mass and bone density loss, hepatomegaly, menstrual irregularities in women and gynecomastia in men, impaired mental status, itching, kidney function failure and muscle wasting can be developed.

### Acute on Chronic Liver Failure (ACLF)

ACLF is also an important hepatic condition observed in patients with known chronic liver disease who have acute deterioration of the liver function.

ACLF is an abrupt and life-threatening worsening of clinical conditions in patients with advanced cirrhosis or with cirrhosis due to a chronic liver disease. Three major features characterize this syndrome: it generally occurs in the context of intense systemic inflammation, frequently develops in close temporal relationship with proinflammatory precipitating events (e.g., infections or alcoholic hepatitis), and is associated with single- or multiple-organ failure affecting liver, kidneys, brain, coagulation and/or cardiovascular functions. Organ failures are identified with the use of a modified Sequential Organ Failure Assessment score (the EASL-CLIF Consortium organ failure scoring system), which considers the function of the liver, kidney, and brain, as well as coagulation, circulation, and respiration, allowing stratification of patients in subgroups with different risks of death. According to the number of organ failures at diagnosis, patients were stratified into four prognostic grades (no acute-on-chronic liver failure and acute-on-chronic liver failure grades 1, 2, and 3). Predisposition to ACLF is correlated to the severity (i.e. fibrosis advancement up to cirrhosis) and etiology of underlying chronic liver disease. Whatever the etiology of the chronic liver disease, compensated cirrhosis is the main condition associated with development of ACLF. Cholestatic, metabolic liver diseases, chronic viral hepatitis and nonalcoholic steatohepatitis (NASH) are also qualified as underlying chronic liver disease. Alcoholic cirrhosis constitutes 50-70% of all underlying liver diseases of ACLF in Western countries, whereas viral hepatitis-related cirrhosis constitutes about 10-30% of all cases.

The severity of underlying disease can be assessed by the Model for End-Stage Liver Disease (MELD) scores.

ACLF requires a precipitating event that occurs in the setting of cirrhosis and/or chronic liver disease, and progresses rapidly to multiorgan failure with high mortality. The precipitating events may be reactivation of hepatitis B or superimposed viral hepatitis, alcohol, drugs, ischemic, surgery, sepsis or idiopathic.

During the onset of the disease, bacterial translocation may play a pivotal role in the progression from compensated to decompensated liver cirrhosis (as marked by the development of ascites, variceal bleeding, encephalopathy) via the systemic inflammatory response syndrome. However, about 40% of patients with ACLF have no precipitating events.

Host response determines the severity of injury. Inflammation and neutrophil dysfunction are of major importance in the pathogenesis of ACLF, and a prominent pro-inflammatory cytokine profile causes the transition from stable cirrhosis to ACLF. In these patients, an inflammatory response may lead to immune dysregulation, which may predispose to infection that would then further aggravate a pro-inflammatory response resulting in a vicious cycle. Cytokines are believed to play an important role in ACLF. Elevated serum levels of several cytokines, including tumor necrosis factor (TNF)-α, sTNF-αR1, sTNF-αR2, interleukin (IL)-2, IL-2R, IL-4, IL-6, IL-8, IL-10, and interferon-α, have been described in patients with ACLF.

Hyperbilirubinemia is almost invariably present and jaundice is considered an essential criterion of ACLF. Various authors have used different cutoff levels of jaundice, varying from a serum bilirubin of 6-20 mg/dL. Besides jaundice, another hallmark of liver dysfunction is coagulopathy. Coagulation tests are usually abnormal in cirrhotic patients due to impaired synthesis and increased consumption of coagulation factors. Ongoing liver injury culminates in an inexorable downward spiral and death.

The most common organ to fail besides liver is the kidney. Renal failure may be categorized into four types: hepatorenal syndrome, parenchymal disease, hypovolemia-induced and drug-induced renal failure. Bacterial infection (such as spontaneous bacterial peritonitis) is the most common precipitating cause of renal failure in cirrhosis, followed by hypovolemia (secondary to gastrointestinal bleeding, excessive diuretic treatment).

Hepatic encephalopathy (HE) is one of the common manifestations of ACLF. HE may be a precipitating factor or a consequence of ACLF. Ammonia is central to the pathogenesis of HE. Indeed, multiple studies have highlighted that hyperammonemia plays a critical role in the development of HE in patients with liver cirrhosis and other liver diseases. Due to liver failure, a large amount of serum ammonia escapes liver metabolism and can reach brain where such high ammonia concentrations are closely related to a high incidence of cerebral edema and herniation.

In addition, brain swelling is an important feature of ACLF, similar to the situation in ALF.

One of the hallmark of ACLF is cardiovascular collapse akin to that in patients with ALF. This cardiovascular abnormality is associated with an increased risk of death, particularly in those patients who present renal dysfunction.

Respiratory complications in ACLF can be categorized as acute respiratory failure (e.g., pneumonia) and those that arise as a consequence of cirrhosis (e.g., portopulmonary hypertension and hepatopulmonary syndrome). Patients with cirrhosis are at increased risk of pneumonia.

Patients with ACLF have a statistically higher mortality rate at the same MELD score than patients without ACLF. Regardless of the precipitating event, the final common pathway leading to acute deterioration of liver function and multiorgan failure appears to be an exaggerated activation of systemic inflammation, which is then followed by a period of immune system paralysis. The initial cytokine storm is responsible for profound alterations in macrocirculation, microcirculation, and disruption of normal organ function, resulting in multiorgan failure.

Early interventions to reduce or correct injury are crucial. Management of ACLF is currently based on the supportive treatment of organ failures, mainly in an intensive care setting.

However, the proportion of cases with previous episodes of acute decompensation (development of ascites, encephalopathy, gastrointestinal hemorrhage, bacterial infection) is very frequent in patients with ACLF. Indeed, the appearance of liver failure in a patient with cirrhosis represents a decisive time point in terms of medical management since this condition is frequently associated with rapidly evolving multiorgan dysfunction. The lack of liver detoxification, metabolic and regulatory functions and altered immune response lead to life-threatening complications, such as renal failure, increased susceptibility to infection, hepatic coma and systemic hemodynamic dysfunction. Moreover, only 20% of patients with advanced cirrhosis can be treated with liver transplantation.

There is a need for an adequate treatment or prevention of liver failure, in particular of ACLF, ALF and cirrhosis, more particularly of decompensated cirrhosis.

NTZ (nitazoxanide), first described in 1975, was shown to be highly effective against anaerobic protozoa, helminths, and a wide spectrum of microbes including both anaerobic and aerobic bacteria. NTZ can also confer antiviral activity and was also shown to have broad anticancer properties by interfering with crucial metabolic and prodeath signaling pathways.

A pilot study on HE patients given NTZ and lactulose provided results with regard to amelioration of the clinical picture. Patients showed improvement in mental status and the drug was well-tolerated (Elrakaybi et al., The clinical effects of nitazoxanide in hepatic encephalopathy patients: a pilot study, IJPSR, 2015; Vol. 6(11): 4657-4667). However, the authors of this study did not report improvement in liver function, or any other organ function. Furthermore, ammonia levels were not improved after NTZ administration.

It is herein surprisingly shown that NTZ can be used for the treatment of a subject having or at risk of having a liver failure.

### SUMMARY OF THE INVENTION

The invention stems from the surprising observation that NTZ protects hepatocytes from ammonia-induced toxicity and improves liver functions. Moreover, it is herein surprisingly shown that NTZ prevents decompensation in an animal model of ACLF.

Accordingly, the invention relates to nitazoxanide (NTZ), tizoxanide (TZ), tizoxanide glucuronide (TZG), or a pharmaceutically acceptable salt thereof, for use in a method of treatment or prevention of a liver failure in a subject in need thereof. In a particular embodiment, the liver failure is ACLF, ALF or cirrhosis, in particular compensated or decompensated cirrhosis, more particularly decompensated cirrhosis. In a particular embodiment, the liver failure is ACLF or ALF.

In a particular embodiment, the invention relates to NTZ, TZ, TZG, or a pharmaceutically acceptable salt thereof, for use in a method of treatment of a liver failure selected from ACLF and ALF. In a particular embodiment, the invention relates to NTZ, TZ, TZG, or a pharmaceutically acceptable salt thereof, for use in a method of treatment of ACLF.

The invention further relates to NTZ, TZ, TZG, or a pharmaceutically acceptable salt thereof, for use in a method of treatment or prevention of liver failure due to ACLF, ALF, cirrhosis or cirrhosis decompensation, in a subject in need thereof. In a particular embodiment, NTZ, TZ, TZG, or a pharmaceutically acceptable salt thereof, are used in a method of treatment or prevention of liver failure due to ACLF or ALF.

In a particular embodiment, the subject is at risk of ACLF, ALF or cirrhosis, in particular of decompensated cirrhosis (also herein referred to as a “subject at risk”).

In a particular embodiment, the subject has ACLF. Accordingly, NTZ, TZ, TZG, or a pharmaceutically acceptable salt thereof, is for use in a method of treatment of ACLF.

In yet another embodiment the subject has ALF. Accordingly, NTZ, TZ, TZG, or a pharmaceutically acceptable salt thereof, is for use in a method of treatment of ALF. In a particular embodiment, the method is for the treatment of drug-induced ALF, in particular of acetaminophen-induced ALF.

In yet another embodiment, the subject has cirrhosis. In another particular embodiment, the subject has compensated or decompensated cirrhosis. In a further embodiment, the subject has decompensated cirrhosis. In a further particular embodiment, the subject has compensated cirrhosis, and the method is for the prevention of the progression from compensated cirrhosis to decompensated cirrhosis.

In yet another embodiment, NTZ, TZ, TZG, or a pharmaceutically acceptable salt thereof, is for use in a method of prevention of decompensated cirrhosis, or of reversion of decompensated cirrhosis to compensated cirrhosis.

In another embodiment, the subject has ACLF and cirrhosis. In another particular embodiment, the subject has ACLF and compensated or decompensated cirrhosis. In a further embodiment, the subject has ACLF and decompensated cirrhosis. In a further particular embodiment, the subject has ACLF and compensated cirrhosis, and the method is for the prevention of the progression from compensated cirrhosis to decompensated cirrhosis.

In yet another embodiment, the subject at risk of ACLF has cirrhosis, in particular compensated or decompensated cirrhosis, more particularly decompensated cirrhosis.

In yet another particular embodiment, the subject has ACLF with a high risk of death less than 28 days after hospital admission.

In a particular embodiment, the subject has ACLF grade 1, 2 or 3 according to the CANONIC study of the EASL-CLIF consortium, in particular ACLF grade 2 or 3.

In a particular embodiment, NTZ, TZ(G) or a pharmaceutically acceptable salt thereof, is for use in a method of preventing ACLF in a subject having cirrhosis. In a further particular embodiment, the subject has alcoholic cirrhosis. In another particular embodiment, the subject has compensated alcoholic cirrhosis. In a further particular embodiment, the subject has decompensated alcoholic cirrhosis.

In another embodiment, NTZ, TZ(G) or a pharmaceutically acceptable salt thereof, is for use in a method of preventing ACLF in a subject having chronic liver disease. In a particular embodiment, the subject has a chronic liver disease with or without cirrhosis.

In a particular embodiment, the subject has a chronic liver disease with cirrhosis. In a further particular embodiment, the cirrhosis is a compensated or decompensated cirrhosis, more particularly a decompensated cirrhosis.

In another particular embodiment, NTZ, TZ(G) or pharmaceutically acceptable salt thereof is for use in a method to improve liver function in a subject having a liver failure selected in the group consisting of ACLF, ALF and cirrhosis, in particular compensated or decompensated cirrhosis, more particularly decompensated cirrhosis. In another particular embodiment, the method is to improve liver function in a subject having a liver failure selected in the group consisting of ACLF and ALF.

In another particular embodiment, NTZ, TZ(G) or pharmaceutically acceptable salt thereof is for use in a method to improve liver detoxification function in a subject having a liver failure selected in the group consisting of ACLF, ALF and cirrhosis, in particular compensated or decompensated cirrhosis, more particularly decompensated cirrhosis.

In a particular embodiment, the method is to improve liver detoxification function in a subject having a liver failure selected in the group consisting of ACLF and ALF.

In another particular embodiment, NTZ, TZ(G) or pharmaceutically acceptable salt thereof is for use in a method to prevent liver decompensation in a subject having ACLF.

In a particular embodiment, the subject has ACLF combined to at least one precipitating event, such as an infection (e.g. a viral, fungal, or bacterial infection) or alcohol hepatitis, sepsis, poisoning, visceral bleeding and drug-induced liver insufficiency (DILI).

In yet another particular embodiment, the subject has ACLF provoked by a hepatic precipitating condition (e.g. alcohol), an extrahepatic precipitating conditions (e.g. a viral, fungal, or bacterial infection, sepsis, virus reactivation), or both.

In another particular embodiment, the subject is at risk of ACLF, with a precipitating event. For example, the subject can have a chronic liver disease with a precipitating event. In a particular embodiment, the subject has a chronic liver disease with a precipitating event, with or without cirrhosis. In a particular embodiment, the subject has a chronic liver disease with a precipitating event and cirrhosis. In a further particular embodiment, the cirrhosis is a compensated or decompensated cirrhosis, more particularly a decompensated cirrhosis.

In yet another embodiment, NTZ, TZ(G) or a pharmaceutically acceptable salt thereof, is for use in a method of preventing extrahepatic organ failure in a subject having cirrhosis, in particular in a subject having compensated or decompensated cirrhosis, in particular decompensated cirrhosis. In a particular embodiment, the prevented extrahepatic organ failure is kidney failure. In another particular embodiment, the prevented extrahepatic organ failure is brain failure. In another particular embodiment, the prevented extrahepatic organ failure is cardiac failure. In another particular embodiment, the prevented extrahepatic organ failure is pulmonary failure.

In yet another embodiment, NTZ, TZ(G) or a pharmaceutically acceptable salt thereof, is for use in a method of preventing extrahepatic organ failure in a subject having chronic liver disease with cirrhosis, in particular in a subject having compensated or decompensated cirrhosis, in particular decompensated cirrhosis. In a particular embodiment, the prevented extrahepatic organ failure is kidney failure. In another particular embodiment, the prevented extrahepatic organ failure is brain failure. In another particular embodiment, the prevented extrahepatic organ failure is cardiac failure. In another particular embodiment, the prevented extrahepatic organ failure is pulmonary failure.

In yet another embodiment, NTZ, TZ(G) or a pharmaceutically acceptable salt thereof, is for use in a method of treating extrahepatic organ failure in a subject having ACLF. In a particular embodiment, the treated extrahepatic organ failure is kidney failure. In another particular embodiment, the treated extrahepatic organ failure is brain failure. In another particular embodiment, the prevented extrahepatic organ failure is cardiac failure. In another particular embodiment, the prevented extrahepatic organ failure is pulmonary failure.

In yet another embodiment, NTZ, TZ(G) or a pharmaceutically acceptable salt thereof, is for use in a method of preventing extrahepatic organ failure in a subject having ACLF. In a particular embodiment, the prevented extrahepatic organ failure is kidney failure. In another particular embodiment, the prevented extrahepatic organ failure is brain failure. In another particular embodiment, the prevented extrahepatic organ failure is cardiac failure. In another particular embodiment, the prevented extrahepatic organ failure is pulmonary failure.

In a further particular embodiment, NTZ, TZ(G) or a pharmaceutically acceptable salt thereof, is for use in a method of protecting the subject from ammonia-induced injury, said subject having or being at risk of a liver failure selected in the group consisting of ACLF, ALF and decompensated cirrhosis.

In a further particular embodiment, NTZ, TZ(G) or a pharmaceutically acceptable salt thereof, is for use in a method of treating or preventing HE. In a particular embodiment, the method is for treating or preventing HE, and further preventing brain edema. In a particular embodiment, NTZ, TZ(G) or a pharmaceutically acceptable salt thereof, is for use in a method of treating or preventing HE in a subject having a liver failure, such as a liver failure selected in the group consisting of ACLF, ALF and cirrhosis, in particular compensated or decompensated cirrhosis, more particularly decompensated cirrhosis. In yet another particular embodiment, NTZ, TZ(G) or a pharmaceutically acceptable salt thereof, is for use in a method of treating or preventing HE in a subject at risk of having a liver failure, such as at risk of having a liver failure selected in the group consisting of ACLF, ALF and cirrhosis, in particular compensated or decompensated cirrhosis, more particularly decompensated cirrhosis. In yet another embodiment, NTZ, TZ(G) or a pharmaceutically acceptable salt thereof, is for use in a method of preventing HE in a subject having a liver failure, such as a liver failure selected in the group consisting of ACLF, ALF and cirrhosis, in particular compensated or decompensated cirrhosis, more particularly decompensated cirrhosis. In a further particular embodiment, NTZ, TZ(G) or a pharmaceutically acceptable salt thereof, is for use in a method of treating or preventing HE by protecting the brain from ammonia-induced injury, in a subject having or being at risk of a liver failure selected in the group consisting of ACLF, ALF and cirrhosis, in particular compensated or decompensated cirrhosis, more particularly decompensated cirrhosis.

In a further particular embodiment, NTZ, TZ(G) or a pharmaceutically acceptable salt thereof, is for use in a method of treating or preventing brain edema. In a particular embodiment, NTZ, TZ(G) or a pharmaceutically acceptable salt thereof, is for use in a method of treating or preventing brain edema in a subject having a liver failure, such as a liver failure selected in the group consisting of ACLF, ALF and cirrhosis, in particular compensated or decompensated cirrhosis, more particularly decompensated cirrhosis. In yet another particular embodiment, NTZ, TZ(G) or a pharmaceutically acceptable salt thereof, is for use in a method of treating or preventing brain edema in a subject at risk of having a liver failure, such as at risk of having a liver failure selected in the group consisting of ACLF, ALF and cirrhosis, in particular compensated or decompensated cirrhosis, more particularly decompensated cirrhosis. In yet another embodiment, NTZ, TZ(G) or a pharmaceutically acceptable salt thereof, is for use in a method of preventing brain edema in a subject having a liver failure, such as a liver failure selected in the group consisting of ACLF, ALF and cirrhosis, in particular compensated or decompensated cirrhosis, more particularly decompensated cirrhosis. In a further particular embodiment, NTZ, TZ(G) or a pharmaceutically acceptable salt thereof, is for use in a method of treating or preventing brain edema by protecting the brain from ammonia-induced injury, in a subject having or being at risk of a liver failure selected in the group consisting of ACLF, ALF and cirrhosis, in particular compensated or decompensated cirrhosis, more particularly decompensated cirrhosis.

### BRIEF DESCRIPTION OF THE DRAWINGS

FIG. 1 is a graph showing dead cell protease activity after treatment with or without NTZ. The protective effect of NTZ on ammonium chloride (NH₄Cl-induced mortality was assessed in HepG2 cells. A dose response of NTZ was evaluated upon low (60 mM) and high (120 mM) concentrations of NH₄Cl. Cell mortality was estimated by protease activity measurement. Bar graph representing mean and standard deviation. § p<0.05 using non-parametric Mann-Whitney test in comparison to the control without NH4Cl. [symbol] , [symbol] [symbol] , [symbol] [symbol] [symbol] p<0.05, p<0.01, p<0.001 using non-parametric Kruskal-Wallis test in comparison to the same dose of NH4Cl without NTZ (NTZ 0 μM).

FIG. 2 provides representative pictures of liver sections of TAA induced cirrhosis in rats. Hematoxylin, eosin and safranin stained liver sections of rat livers after 16 weeks of TAA administration treated or not with NTZ for the last 4 weeks. The fibrous septa (in black) surround the hepatic nodules (in grey) as a hallmark of cirrhosis. Portal tract and vessels are depicted in white.

FIG. 3 is a graph representing plasma total bilirubin level in cirrhotic rats treated with or without NTZ. Bar graph representing mean and standard deviation. §, § §, § § §: p<0.05, p<0.01, p<0.001 using non-parametric Mann-Whitney test in comparison to the TAA control.

FIG. 4 is a graph representing plasma albumin level in cirrhotic rates treated with or without NTZ. Bar graph representing mean and standard deviation. *, **: p<0.05, p<0.01 using Student t-test in comparison to the TAA control.

FIG. 5 is a schematic representation of a protocol of ACLF induction and NTZ treatment.

FIG. 6 is a graph showing the survival curves after LPS injection in cirrhotic rats treated or not (vehicle, veh) with NTZ. P=0.058 for the comparison of the survival curves using Mantel-Cox test.

FIG. 7 is a graph showing plasma total bile acids in cirrhotic rats treated or not (vehicle, veh) with NTZ after ACLF induction. * p<0.05 using a Student t-test.

FIG. 8 is a graph showing that NTZ reduces brain edema in rats with BDL+LPS-induced ACLF. Bar graph representing mean and standard deviation. * p<0.05, ** p<0.01 using Student T test.

FIG. 9 is a graph showing that NTZ improves renal function in rats with BDL+LPS-induced ACLF. Bar graph representing mean and standard deviation. #p<0.05, ##p<0.01 using Mann-Whitney test.

FIG. 10 is a graph showing that NTZ reduces plasma AST after acetaminophen intoxication. Bar graph representing mean and standard deviation. * p<0.05, ** p<0.01 using ANOVA and Dunnett's multiple comparisons test.

FIG. 11 is a graph showing that NTZ treatment prevents from kidney injury. Bar graphs representing mean and standard deviation of plasma creatinine, urea and cystatin C concentrations. * p<0.05, ** p<0.01 using Student T test with Welsh correction.

FIG. 12 is a graph showing that NTZ greatly reduces ALT and AST levels in animals treated with CCl4+LPS. Experimental results are expressed as mean±standard deviation and plotted as bar graphs. Comparison between groups was tested using a Student T test for all variables that follows a normal distribution (#: p<0.05; ##: p<0.01; ###: p<0.001). A Welsh test was applied in case of different variances between groups ( [symbol] : p<0.05; [symbol] [symbol] : p<0.01; [symbol] [symbol] [symbol] : p<0.001). A log-transformation was applied for plasma ALT and AST in all samples to obtain a normal data distribution.

FIG. 13 is a graph showing that NTZ greatly reduces the level of GGT in animals treated with CCl4+LPS. Experimental results are expressed as mean±standard deviation and plotted as bar graphs. A non-parametric Mann-Whitney test was applied ($: p<0.05; $$: p<0.01; $$$: p<0.001).

FIG. 14 is a graph showing that NTZ prevents LPS-induced alteration of hepatic function, as shown by its action of the reduction of total bilirubin and restoration of albumin production. Experimental results are expressed as mean±standard deviation and plotted as bar graphs. A Welsh test was applied in case of different variances between groups ( [symbol] : p<0.05; [symbol] [symbol] : p<0.01; [symbol] [symbol] [symbol] : p<0.001). A non-parametric Mann-Whitney test was applied ($: p<0.05; $$: p<0.01; $$$: p<0.001).

FIG. 15 is a graph showing that NTZ presents LPS-induced renal impairment, as evidenced by the reduction of plasma creatinine, urea and cystatin C. Experimental results are expressed as mean±standard deviation and plotted as bar graphs. A non-parametric Mann-Whitney test was applied ($: p<0.05; $$: p<0.01; $$$: p<0.001).

FIG. 16 is a graph showing that NTZ has an inflammatory effect in an ACLF model, as evidenced by its ability to reduce the level of LPS-induced serum IFNγ. Experimental results are expressed as mean±standard deviation and plotted as bar graphs. Comparison between groups was tested using a Student T test for all variables that follows a normal distribution (#: p<0.05; ##: p<0.01; ###: p<0.001).

### DETAILED DESCRIPTION OF THE INVENTION

The subject in need of the treatment provided herein is a patient with a liver failure. In a particular embodiment, the subject is a patient with a liver failure selected in the group consisting of ACLF, ALF and cirrhosis, such as compensated or decompensated cirrhosis, more particularly decompensated cirrhosis.

Alternatively, the subject in need of the treatment provided herein is a patient at risk of a liver failure selected from ACLF, ALF and cirrhosis, in particular compensated or decompensated cirrhosis, more particularly decompensated cirrhosis. In particular, the subject may be a patient at risk of cirrhosis decompensation or ACLF due to a chronic liver disease.

The term “subject” or “patient” as used herein refers to a mammal, preferably a human.

ACLF is a multiorgan syndrome that generally in subjects with cirrhosis, with at least one organ failure and with high short-term mortality rate. ACLF develops in patients with chronic liver disease in response to sur-imposed precipitating factors.

In a particular embodiment, the subject suffers from a chronic liver disease with cirrhosis and is at risk of developing ACLF.

The term “chronic liver disease” is used herein to refer to liver diseases associated with a chronic liver injury regardless of the underlying cause. A chronic liver disease may result, for example, from alcohol abuse (alcoholic hepatitis), viral infectious (e.g. viral hepatitis A, B, C, E) or autoimmune processes (autoimmune hepatitis), nonalcoholic steatohepatitis (NASH), cancer or chronic exposure to mechanical or chemical injury to the liver, or from cancer. Chemical injury to the liver can be caused by a variety of substances, such as toxins, alcohol, carbon tetrachloride, trichloroethylene, iron or medications.

In a particular embodiment, the subject has a chronic liver disease with cirrhosis. In a particular embodiment, the subject has cirrhosis consecutive to:
alcohol abuse,
viral hepatitis (such as a viral hepatitis resulting from hepatitis A, B, C, D, E, or G virus infection),
use of medication,
metabolic disease,
a biliary disease,
primary biliary cholangitis,
primary sclerosing cholangitis, or
NASH.

The present invention is particularly suitable for the prevention of the recurrence or management of ACLF.

In a particular embodiment, the subject with cirrhosis decompensation or ACLF, shows a high MELD score. The term “MELD score” or “Model for End-Stage Liver Disease” as used herein refers to a scoring system for assessing the severity of liver dysfunction. MELD uses the patient's values for serum bilirubin, serum creatinine and the international ratio for prothrombin time (INR) to predict survival. It is calculated according to the following formula:

MELD=3.78[Ln serum bilirubin (mg/dL)]+11.2[Ln INR]+9.57[Ln serum creatinine (mg/dL)]+6.43 wherein, Ln means Napierian logarithm.

Bilirubin is the yellow breakdown product of normal heme catabolism. Bilirubin is excreted in bile and urine. Most bilirubin (70-90%) is derived from hemoglobin degradation and, to a lesser extent, from other hemoproteins. In serum, bilirubin is usually measured as both direct bilirubin and total bilirubin. Direct bilirubin correlates with conjugated bilirubin and it includes both the conjugated bilirubin and bilirubin covalently bound to albumin. Indirect bilirubin correlates to unconjugated bilirubin. The serum bilirubin level can be measured by any suitable method known in the art. Illustrative non-limitative examples of methods for determining serum bilirubin include methods using diazo reagent, methods with DPD, methods with bilirubin oxidase or by means of direct spectrophotometric determination of bilirubin. Briefly, the method for determining the levels of bilirubin in serum with diazo reagents is based on the formation of azobilirubin, which acts as indicator by means of addition of a mixture of sufanilic acid and sodium nitrite. The method based in determining serum bilirubin with DPD is based on the fact that bilirubin reacts with 2,5-dichlorobenzenediazonium salt (DPD) in 0.1 mol/HCl forming azobilirubin with maximal absorbance at 540-560 nm. The staining intensity is proportional to the concentration of bilirubin. Unconjugated bilirubin reacting in the presence of detergent (e.g. Triton TX-100) is determined as total bilirubin whereas only conjugated bilirubin reacts in the absence of detergent. The method for determining the serum level of bilirubin with bilirubin oxidase is based on the reaction catalyzed by the enzyme bilirubin oxidase which oxidizes bilirubin to biliverdin with maximal absorbance at 405-460 nm. The concentration of bilirubin is proportional to the measured absorbance. The concentration of total bilirubin is determined by the addition of sodium dodecyl sulfate (SDS) or sodium cholate which evokes the separation of unconjugated bilirubin from albumin and a reaction of precipitation. The level of serum bilirubin can also be determined by direct spectrophotometric at 454 nm and 540 nm. The measurement at these two wavelengths is used to diminish the hemoglobin interference.

The term “international ratio for prothrombin time”, or “INR” as used herein, refers to a parameter used to determine the clotting tendency of blood. The INR is the ratio of a patient's prothrombine time to a normal (control) sample, raised to the power of the ISI value for the analytical system used. Prothrombin time (PT) measures factors I (fibrinogen), II (prothrombin), V, VII and X and it is used in conjunction with the activated partial thromboplastin time. The prothrombin time is the time it takes plasma to clot after addition of tissue factor. This measures the extrinsic pathway of coagulation. The INR standardizes the results of prothrombine time and is calculated by the following formula: INR=(PTtest/PTnormal)<ISI>

The ISI value of the formula is the International Sensitive Index for any tissue factor and it indicates how a particular batch of tissue factor compares to an international reference tissue factor. The ISI is usually between 1.0 and 2.0.

The value of MELD score correlates strongly with short-term mortality, the lower the value of MELD score the lower the mortality and the higher the value of the MELD score, the higher the mortality. Thus, a patient having low MELD score, for example a MELD lower than 9, has about 1.9% 3-month mortality whereas patients having high MELD score, for example a MELD score of 40 or more, have about 71.3% 3-month mortality.

The term “high MELD score” as used herein, refers to a patient having a MELD score higher than 9, for example, at least 10, at least 15, at least 19, at least 20, at least 25, at least 29, at least 30, at least 35, at least 39, at least 40, at least 45 or more. In a particular embodiment, the present invention is applied to a subject having a MELD score higher than 20.

In another particular embodiment, the patient to be treated shows impairment of kidney function. The term “impairment of kidney function”, also known as “impairment of renal function”, “renal impairment (disorder)”, “renal insufficiency”, “renal impairment” and “renal failure”, as used herein, refers to a medical condition in which the kidneys fail to adequate filter waste products from the blood. Renal failure is mainly determined by a decrease in glomerular filtration rate, the rate which blood is filtered in the glomeruli of the kidney. In renal failure, there may be problems with increased fluid in the body (leading to swelling), increased acid levels, raised levels of potassium, decreased levels of calcium, increased level of phosphate, and in later stages, anemia.

Subjects having liver failure with HE who can benefit from the present invention may be identified thanks to polymersomes and methods using the same disclosed in WO2019053578.

The term “treatment”, as used herein, relates to both therapeutic measures and prophylactic or preventative measures, wherein the goal is to prevent or slow down (lessen) an undesired physiological change or disorder. Beneficial or desired clinical results include, but are not limited to, alleviation of symptoms, stabilizing pathological state (specifically not worsening), slowing down or stopping the progression of the disease, improving or mitigating the pathological. Particularly, for the purpose of the present invention, treatment is directed to slow the progression of liver damage and reduce the risk of further complications. It can also involve prolonging survival in comparison with the expected survival if the treatment is not received.

In the context of the present invention NTZ, TZ(G), or a pharmaceutically acceptable salt thereof is administered to the subject, in a therapeutically effective amount. In a particular embodiment, NTZ or TZ, or a pharmaceutically acceptable salt thereof is administered. In a further embodiment, the subject is administered with NTZ or a pharmaceutically acceptable salt thereof, in particular with NTZ.

A “therapeutically effective amount” refers to an amount of the drug effective to achieve a desired therapeutic result. A therapeutically effective amount of a drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The effective dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

NTZ, TZ(G) or a pharmaceutically acceptable salt thereof can be formulated in a pharmaceutical composition further comprising one or several pharmaceutically acceptable excipients or vehicles (e.g. saline solutions, physiological solutions, isotonic solutions, etc.), compatible with pharmaceutical usage and well-known by one of ordinary skill in the art.

These compositions can also further comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc.

These compositions can be formulated in the form of injectable suspensions, syrups, gels, oils, ointments, pills, tablets, suppositories, powders, gel caps, capsules, aerosols, etc., eventually by means of galenic forms or devices assuring a prolonged and/or slow release. For this kind of formulations, agents such as cellulose, carbonates or starches can advantageously be used.

NTZ or TZ(G) can be in the form of pharmaceutically acceptable salts particularly acid or base salts compatible with pharmaceutical use. Salts of NTZ and TZ(G) include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

NTZ, TZ(G), or a pharmaceutically acceptable salt thereof may be administered by different routes and in different forms. For example, the compound(s) may be administered via a systemic way, per os, parenterally, by inhalation, by nasal spray, by nasal instillation, or by injection, such as for example intravenously, by intramuscular route, by subcutaneous route, by transdermal route, by topical route, by intra-arterial route, etc. Of course, the route of administration will be adapted to the form of the drug according to procedures well known by those skilled in the art.

In a particular embodiment, the compound is formulated as a tablet. In another particular embodiment, the compound is administered orally.

The frequency and/or dose relative to the administration can be adapted by one of ordinary skill in the art, in function of the patient, the pathology, the form of administration, etc. Typically, NTZ or TZ(G) can be administered at a dose comprised between 0.01 mg/day to 4000 mg/day, such as from 50 mg/day to 2000 mg/day, such as from 100 mg/day to 2000 mg/day; and particularly from 100 mg/day to 1000 mg/day. In a particular embodiment, the NTZ, TZ(G), or a pharmaceutically acceptable salt thereof, is administered at a dose of about 1000 mg/day, in particular at 1000 mg/day. In a particular embodiment, NTZ, TZ(G), or a pharmaceutically acceptable salt thereof, is administered orally at a dose of about 1000 mg/day, in particular at 1000 mg/day, in particular as a tablet. Administration can be performed daily or even several times per day, if necessary. In one embodiment, the compound is administered at least once a day, such as once a day, twice a day, or three times a day. In a particular embodiment, the compound is administered once or twice a day. In particular, oral administration may be performed once a day, during a meal, for example during breakfast, lunch or dinner, by taking a tablet comprising the compound at a dose of about 1000 mg, in particular at a dose of 1000 mg. In another embodiment, a tablet is orally administered twice a day, such as by administering a first tablet comprising the compound at a dose of about 400 mg, about 500 mg or about 600 mg, in particular at a dose of 500 mg, during one meal, and administering a second tablet comprising the compound at a dose of about 500 mg, in particular at a dose of 500 mg, during another meal the same day.

In the context of the present invention, the term “about” applied to a numerical value means the value +/−10%. For the sake of clarity, this means that “about 100” refers to values comprised in the 90-110 range. In addition, in the context of the present invention, the term “about X”, wherein X is a numerical value, also discloses specifically the X value, but also the lower and higher value of the range defined as such, more specifically the X value.

Suitably, the course of treatment with NTZ, TZ(G) or a pharmaceutically acceptable salt thereof is for at least 1 week, in particular for at least 2, 3, 4, 5, 6, 7, 8, 9, 10, 15, 20 or 24 weeks or more. In a particular embodiment, the course of treatment is for at least 1 month, at least 2 months or at least 3 months. In a particular embodiment, the course of treatment is for at least 1 year, or more depending on the condition of the subject being treated.

In a particular embodiment, NTZ, TZ(G), or a pharmaceutically acceptable salt thereof (“the drug”), is for use as the sole active ingredient for the treatment or prevention disclosed herein.

In yet another embodiment, the drug is for use in a combination therapy.

In a particular embodiment, the drug is for use in combination with therapy against a precipitating event.

In a particular embodiment, the drug is for use in combination with therapy against a hepatic precipitating condition or extrahepatic precipitating condition.

In a particular embodiment, the precipitating event is a bacterial, fungal or viral infection. Accordingly, the drug can be combined with an antimicrobial or antiviral agent. The most suitable agent will be selected depending on the organism or virus responsible for the invention, as is well known in the art. In a particular embodiment, the precipitating event is hepatitis B virus reactivation. In that case, the drug can be combined with nucleoside or nucleoside analogues. Illustrative antiviral drugs include, without limitation, tenofovir, tenofovir alafenamide and entecavir.

In another particular embodiment, the precipitating event is acute variceal hemorrhage. Accordingly, the drug can be combined with a vasoconstrictor such as terlipressin, somatostatin, or analogues such as octreotide or vapreotide, in particular octreotide. Such treatment may accompany endoscopic therapy (preferably endoscopic variceal ligation, performed at diagnostic endoscopy less than 12 hours after admission). Short-term antibiotic prophylaxis, such as with ceftriaxone, can also be implemented.

In another particular embodiment, the precipitating event is alcoholic hepatitis. Accordingly, the drug can be combined with prednisolone, which is indicated for patients with severe alcoholic hepatitis.

In another particular embodiment, the drug is for use in combination with a supportive therapy.

In a particular embodiment, the supportive therapy is a cardiovascular support. For example, the drug can be combined with a therapy for acute kidney injury, such as withdrawal of diuretics or volume expansion (with intravenous albumin). The drug may also be combined with a vasoconstrictor, such as terlipressin or norepinephrine, in particular if there is no response to volume expansion.

In a particular embodiment, the supportive therapy is a treatment of encephalopathy. For example, the drug can be combined with lactulose. Optionally, lactulose therapy can be further completed with the administration of enemas to clear the bowel. In case the subject has severe hepatic encephalopathy refractory to lactulose, albumin dialysis may be used. In yet another particular embodiment, the drug can be combined with rifaximin. In a further embodiment, the drug can be combined with lactitol.

In a further particular embodiment, the drug is for use in combination with liposomal-based toxin scavengers, such as ammonia scavengers. In particular, the liposomal based toxin scavenger may be a liposomal-based intraperitoneal fluid, which may be used, in particular, to enhance clearance of ammonia, in particular ammonia accumulated during decompensated liver cirrhosis.

The intraperitoneal administration of vesicles (e.g. liposomes) with a remote loading capacity (e.g. transmembrane pH-gradient liposomes) have been described as an interesting approach for the treatment of drug overdose and intoxications to endogenous metabolites (e.g. hyperammonemia) (Forster et al. Sci Transl Med 2014; 6: 258ra141). As mentioned above, hyperammonemia is associated with HE. Combining NTZ, TZ(G), or a pharmaceutically acceptable salt thereof, with such intraperitoneal administration of liposomal based toxin scavengers can therefore be advantageous in the context of the present invention.

In particular, WO 2014/023421 describes liposome vesicles for use in the peritoneal dialysis of patients suffering from endogenous or exogenous toxicopathies, in particular from hyperammonemia, wherein the pH within the liposomes differs from the pH in the peritoneal cavity and wherein the pH within the liposome results in a liposome-encapsulated charged toxin. The liposome formulation may comprise vesicles of various nature, compositions, sizes, and characteristics, enclosing an aqueous medium of diverse compositions, pH and osmotic strength. In preferred embodiments liposome-like vesicles are made from polymers and comprise no lipids, for which reason they are formally not considered liposomes and are called polymersomes. These polymersomes can be administered in any form or mode which makes the liposomes or liposome like vesicles, e.g. polymersomes or niosomes, bioavailable in the peritoneal cavity. It is possible to add a step of extracting the liposomes from the abdominal cavity either subsequently to and/or simultaneously to the administration step. In a particular embodiment, the pH within the liposome vesicles is from 1 to 6.5 and results in a liposome-encapsulated charged toxin, wherein the liposome bilayer comprises as main constituent a natural or synthetic phospholipid, and wherein the diameter size of the liposomes vesicles is larger than 700 nm. In another particular embodiment, the natural or synthetic phospholipid is a long saturated phospholipid. In another particular embodiment, the natural or synthetic phospholipid is a long saturated phospholipid having an alkyl chain of more than 12 carbon atoms. In yet another embodiment, the natural or synthetic phospholipid is a long saturated phospholipid having an alkyl chain of more than 14 carbon atoms. In a further particular embodiment, the natural or synthetic phospholipid is 1,2-Dilauroyl-sn-Glycero-3-Phosphocholine (DLPC); 1,2-Dimyristoyl-sn-Glycero-3-Phosphocholine (DMPC); 1,2-Dipalmitoyl-sn-Glycero-3-Phosphocholine (DPPC); 1,2-Distearoyl-sn-Glycero-3-Phosphocholine (DSPC); 1,2-Dioleoyl-sn-Glycero-3-Phosphocholine (DOPC); 1,2-Dimyristoyl-sn-Glycero-3-Phosphoethanolamine (DMPE); 1,2-Dipalmitoyl-sn-Glycero-3-Phosphoethanolamine (DPPE); 1,2-Distearoyl-sn-Glycero-3-Phosphoethanolamine (DSPE); 1,2-Dioleoyl-SA7-Glycero-3-Phosphoethanolamine (DOPE); 1-Myristoyl-2-Palmitoyl-sn-Glycero-3-Phosphocholine (MPPC); 1-Palmitoyl-2-Myristoyl-sn-Glycero-3-Phosphocholine (PMPC); 1-Stearoyl-2-Palmitoyl-sn-Glycero-3-Phosphocholine (SPPC); 1-Palmitoyl-2-Stearoyl-sn-Glycero-3-Phosphocholine (PSPC); 1,2-Dimyristoyl-sn-Glycero-3-[Phospho-rac-(1-glycerol)] (DMPG); 1,2-Dipalmitoyl-sn-Glycero-3-[Phospho-rac-(1-glycerol)] (DPPG); 1,2-Distearoyl-sn-Glycero-3-[Phospho-rac-(1-glycerol)] (DSPG); 1,2-Dioleoyl-sn-Glycero-3-[Phospho-rac-(1-glycerol)] (DOPG); 1,2-Dimyristoyl-sn-Glycero-3-Phosphate (DMPA); 1,2-Dipalmitoyl-sn-Glycero-3-Phosphate (DPPA); 1,2-Dipalmitoyl-sn-Glycero-3-[Phospho-L-Serine] (DPPS); or L-a-phosphatidylcholine from chicken egg (EPC), or from soy (SPC). In yet another embodiment, the natural or synthetic phospholipid is 1,2-dipalmitoyl-sn-glycero-3-phosphocholine (DPPC). In a further embodiment, the liposome bilayer further comprises a steric stabilizer, wherein the concentration of the steric stabilizer, which can be PEGylated lipids, in the liposome composition can vary between 0 and 30 mol %. In another embodiment, the liposome bilayer comprises between 0.5 and 20 mol % PEGylated lipids. In another embodiment, the PEGylated lipids are 1,2-distearoyl-sn-glycero-3-phosphoethanolamine-N-[methoxy(polyethylene glycol)-2000] (DSPE-PEG). In yet another embodiment, the diameter size of the liposome vesicles is larger than 800 nm. In a further embodiment, the diameter size of the liposome vesicles is from 700 nm to 10 μm. In another embodiment, the pH within the liposome vesicles is from 1.5 to 5, more preferably from 1.5 to 4.

These liposome vesicles can be produced according to methods disclosed in WO 2014/023421 and WO 2016/177741.

WO 2018/033856 describes transmembrane pH-gradient polymersomes and their use in the scavenging of ammonia and its methylated analogs (e.g., trimethylamine (TMA)). Methods for preparing such polymersomes are also disclosed in WO 2018/033856. NTZ, TZ(G) or a pharmaceutically acceptable salt thereof can be combined to such polymersome as liposomal-based toxin scavengers. In a particular embodiment, the polymersome comprises (a) a membrane, which comprises a block copolymer of poly(styrene) (PS) and poly(ethylene oxide) (PEO), wherein the PS/PEO molecular weight ratio is higher than 1.0 and lower than 4.0; and (b) a core which encloses an acid. In yet another embodiment, the block copolymer is a diblock copolymer. In a further embodiment, the acid is in a concentration that produces a pH between 1 and 6, when the polymersome is hydrated. In an embodiment, the acid is within an aqueous acidic solution. In yet another embodiment, the pH within the aqueous acidic solution is between 1 and 6, in particular between 2 and 5, or preferably between 2 and 4. In another embodiment, the acid is a hydroxy acid, most preferably a citric acid. In another embodiment, the polymersome is prepared by a method comprising mixing an organic solvent containing the copolymer with an aqueous phase containing the acid. In yet another embodiment, the organic solvent is water immiscible or partially water miscible. In an embodiment, the core of the polymersome further encloses ammonia or its methylated analog, the methylated analog being preferably TMA. In yet another embodiment, the polymersome is in a composition comprising at least one pharmaceutically acceptable excipient. In another embodiment, the composition is in liquid, semi solid or solid form.

In a particular embodiment, the supportive therapy is an extracorporeal liver support. For example, an extracorporeal liver-assist device that incorporates hepatocytes can be used. In another embodiment, plasma exchange can be conducted in addition to the administration of the drug as provided herein. In yet another embodiment, the extracorporeal liver support is albumin exchange or endotoxin removal.

The following examples serve to illustrate the invention and must not be considered as limiting the scope thereof.

### EXAMPLES

### Example 1: NTZ Induces the Expression of Glutamine Synthetase Involved in Ammonia Detoxification

### Preclinical Model

Liver injury was induced in 5-6 week-old male C57Bl/6 mice (Janvier Labs, France) by feeding them a cholesterol (1%) supplemented choline-deficient L-amino acid-defined diet (CDAA/c) (Ssniff, Germany) for 12 weeks (n=12). Some animals received from day 1 and throughout the study CDAA/c diet supplemented with NTZ (Interchim, France) (100 mg/kg/day) (n=8). Mice receiving choline supplemented L-amino acid-defined (CSAA) diet served as additional controls (n=6).

All animal procedures were performed according to standard protocols and in accordance with the standard recommendations for the proper care and use of laboratory animals.

### Transcriptomic Analysis

Total RNA was isolated from mice livers using Nucleospin® 96 RNA kit (ref 740709.4, Macherey Nagel, France) (n=5). Upon measurement of RNA samples concentration by Multiskan™ GO spectrophotometer (Thermo Scientific, France), the quality was assessed using 2100 Bioanalyser from Agilent (USA). Libraries were prepared using the Illumina TruSeq stranded mRNA LT kit (Ref RS-122-2101, Illumina, USA) and mRNA were sequenced using a NextSeq 500 device (paired-end sequence, 2×75 bp), with a High Output flow cell (ref FC-404-2002—NextSeq® 500/550 High Output Kit v2 (150 cycles), Illumina, USA).

Reads were cleaned using Trimmomatic v.0.36 with the following parameters: SLIDINGWINDOW: 5:20 LEADING: 30 TRAILING: 30 MINLEN: 60 (Bolger et al., Bioinformatics, 2014). Then reads were aligned on the genome reference (Mus musculus GRCm38.90—GenBank assembly accession GCA_000001635.2) with rnacocktail using hisat2 v.2.1.0 as aligner with default parameters (Sahraeian et al. Nature communications 2017).

A count table was produced using featureCounts v1.5.3 with default parameters (Liao Y et al. Bioinformatics 2014).

To identify differentially expressed genes (DE genes), we used R (version 3.4.3) and the DESEq2 library (v. 1.18.1). Gene annotations were retrieved using the AnnotationDbi library (v. 1.40.0). Briefly, the count matrix produced by FeatureCounts was analyzed by the DESeqDataSetFromMatrix( ) function followed by the DEseq( ) function from the DESeq2 library. For each condition (ie comparison NTZ+CDAA/c vs CDAA/c), the fold change and the p-value were retrieved using the results( ) function from DESeq2.

### Results

The analysis of the differentially expressed genes between the hepatic transcriptome of mice treated with NTZ versus the mice receiving only the CDAA/c diet revealed that mRNA expression of GluI is significantly differentially expressed (Table 1).

TABLE 1
Top 10 most significantly genes influenced by NTZ exposure

| | | Average | Fold | | |
|---|---|---|---|---|---|
| | | expression | change | Adjusted | |
| Rank | Symbol | level | (log2) | p value | Gene name |
| 1 | Gsta2 | 4272 | 1.652 | 7.42E−24 | Glutathione S- |
| | | | | | transferase, |
| | | | | | alpha 2 (Yc2) |
| 2 | Abcc4 | 1873 | 1.354 | 3.03E−17 | ATP-binding |
| | | | | | cassette, |
| | | | | | sub-family C |
| | | | | | (CFTR/MRP), |
| | | | | | member 4 |
| 3 | Prodh | 10649 | 0.756 | 1.46E−15 | Proline |
| | | | | | dehydrogenase |
| 4 | Slc1a2 | 18107 | 0.820 | 8.57E−15 | Solute carrier |
| | | | | | family 1 |
| | | | | | (glial high |
| | | | | | affinity |
| | | | | | glutamate |
| | | | | | transporter), |
| | | | | | member 2 |
| 5 | Ldhd | 14329 | 0.659 | 1.35E−14 | Lactate |
| | | | | | dehydrogenase D |
| 6 | Ctsk | 911 | −0.930 | 8.09E−14 | Cathepsin K |
| 7 | Glul | 105162 | 0.672 | 2.61E−13 | Glutamate-ammonia |
| | | | | | ligase (glutamine |
| | | | | | synthetase) |
| 8 | Abat | 21598 | 0.792 | 1.84E−12 | 4-aminobutyrate |
| | | | | | aminotransferase |
| 9 | Sugct | 3484 | 0.664 | 3.14E−12 | Succinyl-CoA |
| | | | | | glutarate-CoA |
| | | | | | transferase |
| 10 | Arhgef26 | 2664 | 1.108 | 4.46E−12 | Rho guanine |
| | | | | | nucleotide |
| | | | | | exchange factor |
| | | | | | (GEF) 26 |

Rank: all differentially expressed genes were sorted according to their adjusted p value
Fold change: NTZ + CDAA/c versus CDAA/c in log2
Adjusted p value: p value of the fold-change adjusted for multiple testing

GluI encodes the glutamine synthetase enzyme involved in hepatic detoxification of ammonia (Zhou et al. Neurochemistry International 2020), a molecule known to induce hepatocellular injury and liver fibrosis. The basal expression level of GluI is very high in the liver of mice (105162 counts in average, Table 1), and its expression is decreased by 40% with the CDAA/c diet compared to CSAA controls (adjusted p=7×10-10). NTZ robustly restores GluI basal expression by inducing it by 1.6-fold in CDAA/c fed mice (adjusted p=2.6×10-13).

Therefore, these data show an important role of NTZ in preventing hepatocellular injury by stimulating ammonia detoxification through glutamine production.

### Example 2: NTZ Protects Hepatocytes from Ammonia-Induced Toxicity

Patients with acute or chronic hepatic diseases often present with hyperammonemia due to alterations in ammonia metabolism and detoxification. This accumulation of ammonia induces in turn hepatocellular damages, formation of scar tissue (fibrosis) and fasten the progression of the disease.

In order to evaluate the effect of NTZ on human hepatocytes that undergo a cellular stress induced by ammonia, the human hepatoblastoma-derived HepG2 cell line (#85011430, ECACC, UK) was cultured in high-glucose DMEM medium supplemented with 10% of fetal bovine serum (FBS), 1% penicillin/streptomycin, 1% sodium pyruvate, 1% of L-glutamine and 1% MEM non-essential amino acids (Gibco, France) in a 5% CO2 incubator at 37° C.

To evaluate the cell tolerance to ammonium chloride (NH₄Cl), 1×105 cells were plated in 96-well plate with a dose range of 0-5 μM of NTZ in the same cell culture medium. After cell adherence (10 hours), cells were washed with PBS and were incubated in the presence of 0, 60, or 120 mM of NH₄Cl (Fluka, France) in DMEM without L-Glutamine and FBS for 14 hours.

Cytotoxicity was measured using CytoTox-Glow™ assay (#G9291, Promega, USA). Briefly, 100 μL of reagent was added per well, and the plates were incubated in the dark at room temperature for 15 min. The CytoTox-Glow™ Assay is a luminescent cytotoxicity assay that measures the relative number of dead cells in cell populations. Luminescence was measured using a Spark microplate reader (#30086376, Tecan, USA). The amount of luminescence (RLU) directly correlates with the percentage of cells undergoing cytotoxic stress.

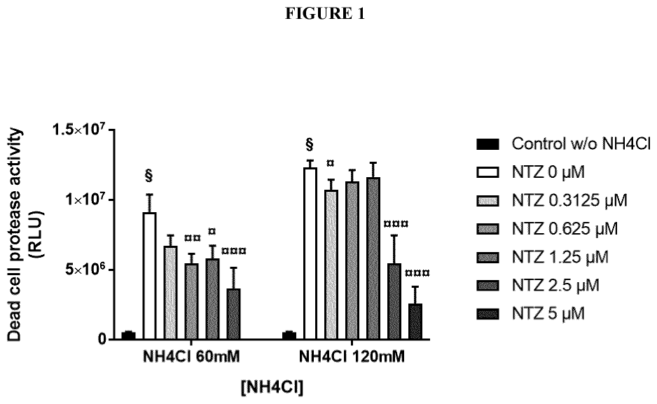

The results are summarized in FIG. 1.

In HepG2, NH₄Cl induced a high mortality rate at both low and high NH₄Cl concentration (18-fold and 23-fold with 60 mM and 120 mM, respectively). Moreover, NTZ reduced the mortality rate in a dose-dependent manner in the 2 conditions of NH₄Cl concentrations (FIG. 1). These results show that NTZ has direct protective effects on liver cells.

### Example 3: NTZ Improves Liver Functions in Cirrhotic Rats

### Preclinical Model of Cirrhosis

Male Sprague Dawley rats (250-275 g) (Janvier, France) received intraperitoneal injections of thioacetamide (TAA) (ref 163678, Sigma) twice a week at the dose of 150 mg/kg during the first week and then at 200 mg/kg during 11 weeks to induce cirrhosis (total induction phase of 12 weeks). Rats were stratified to treatment groups based on mean plasma alpha-2 macroglobulin (a marker of fibrosis) and plasma total bilirubin (a marker of liver function) obtained from sublingual blood sampling. Then, rats received standard diet (n=12) or diet supplemented with NTZ at 30 mg/kg/day (n=10) for an intervention phase of 4 weeks. Rats receiving NaCl intraperitoneal injections during 16 weeks served as additional healthy controls (n=10).

Animals were caged in pairs on a 12:12-hour light-dark cycle, in environmentally controlled animal facilities, with ad libitum access to food and water. The body weight and the food intake were monitored twice a week.

On the last day of treatment, plasma samples were obtained from sublingual blood sampling and rats were sacrificed after a 6 h-fasting period. The liver was rapidly excised for biochemical and histological analyses.

All animal procedures were performed according to standard protocols and in accordance with the standard recommendations for the proper care and use of laboratory animals.

### Tissue Embedding and Sectioning

The liver slices were first fixed for 12-24 hours in formalin 4% solution (Merck Sigma, France). Then, the liver pieces were dehydrated in ethanol solutions (successive baths at 70 and 100% ethanol). The liver pieces were incubated in isopropanol, followed by two baths in liquid paraffin (60° C.). Liver pieces were then put into racks (ref 11670990, Fisher scientific, USA) and filled with Histowax® (ref F/00403, Microm, France) to completely cover the tissue. The paraffin blocks containing the tissue pieces were removed from the racks and stored at room temperature. The liver blocks were cut into 3 μm slices.

### Picrosirius Red Staining

Liver sections were deparaffinized, rehydrated and incubated for 15 minutes in Fast green 0.04% solution (Sigma, cat #F7258-25G). Then, the liver sections were rinsed in acetic acid 0.5% solution and incubated 30 minutes in Picrosirius red 0.1% (Direct red, Alfa Aesar, Germany, cat #621693-25G and Picric acid solution, Sigma, cat #P6744)—Fast Green 0.04% solution. Sections were dehydrated and mounted using the CV Mount medium (Leica, US, cat #14046430011). Collagen proportionate area was assessed by morphometric quantification of picrosirius positive area relative to the liver section area.

### Histological Examinations

Virtual slides were generated using the Pannoramic 250 scanner from 3D Histech (Hungary). For each animal, the fibrosis score was assessed on picrosirius red & fast green stained slices, based on classification of fibrosis into stages according to the localization of collagen depots and the pathological pattern of the hepatic structure, providing an indication of the relative severity and of disease progression. F0: no fibrosis, F1: perisinusoidal or periportal fibrosis, F2: perisinusoidal and portal/periportal fibrosis. F3: bridging fibrosis. F4: cirrhosis.

### Plasma Analyses

The plasmatic concentration of alpha-2 macroglobulin was determined by ELISA using the Abcam kit (cat #ab157730, UK). The intensity of the color measured at 450 nm is proportional to the amount of a 2M bound in the initial step. The sample values are then deduced from the standard curve. Results are expressed in ng/mL.

The concentration of total bilirubin was measured using the Randox kit for Daytona plus automate (Randox, cat #BR 8377). Briefly, the bilirubin is oxidized by vanadate at about pH 2.9 to produce biliverdin. In the presence of detergent and vanadate, both conjugate and unconjugated bilirubin are oxidized. This oxidation reaction causes a decrease in the optical density of the yellow color, which is specific to bilirubin. The decrease in optical density at 450/546 nm is proportional to the total bilirubin concentration in the sample.

The concentration of albumin was measured using the Randox kit for Daytona plus automate (Randox, cat #AB 8301). Briefly, the measurement of albumin is based on its quantitative binding to the indicator 3,3′,5,5′-tetrabromo-m cresol sulphonphthalein (bromocresol green). The album in-BCG-complex absorbs maximally at 578 nm.

### Results

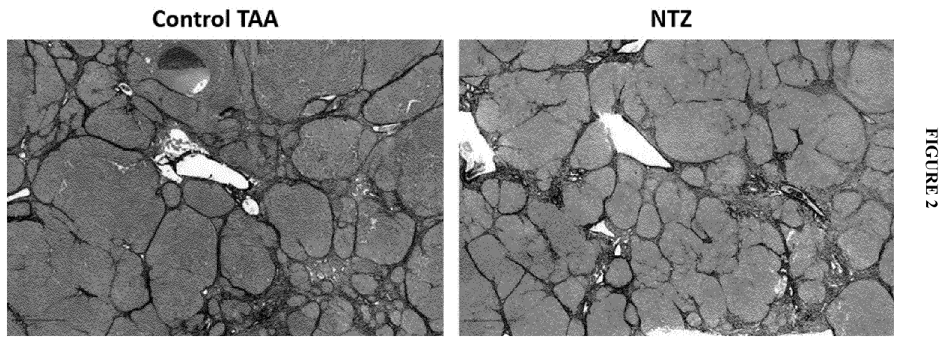

The 16-week TAA administration induced cirrhosis in rats as shown by a histological score of F4 in all animals (FIG. 2).

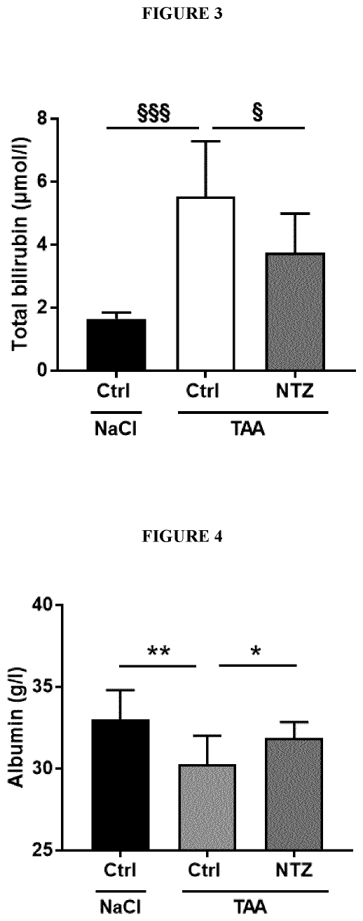

Bilirubin is the result of heme catabolism (mainly derived from hemoglobin in red blood cells). The liver is responsible for clearing the blood of bilirubin. High plasma total bilirubin is a marker of hepatic dysfunction. As expected, plasma total bilirubin increased after 16 weeks of TAA administration. NTZ treatment drastically decreased plasma total bilirubin (FIG. 3).

Albumin is synthesized in the liver. With any liver disease, a fall in plasma albumin reflects decreased synthesis function. As expected, plasma albumin dropped after 16 weeks of TAA administration but was rescued by NTZ treatment (FIG. 4).

Interestingly, these beneficial effects on liver functions were independent of fibrosis as this 4-week intervention treatment with NTZ was too short to observe a significant effect on liver fibrosis (collagen proportionate area of 7.53±0.64% with NTZ versus 7.74±0.38% in the TAA control group, p=0.77). Multivariate analysis confirmed the independent effect of NTZ on liver functions (bilirubin or albumin) in one hand, and fibrosis, on the other.

Taken together, these results show that NTZ has protective effects on liver detoxification and synthesis functions independently of its anti-fibrotic effect, in animal suffering from cirrhosis.

### Example 4: NTZ Prevents Decompensation in an Acute on Chronic Liver Failure (ACLF) Model

### Preclinical Model of ACLF

Male Sprague Dawley rats (175-200 g) (Janvier Labs, France) received intraperitoneal injections of thioacetamide (TAA) 3 times a week at the dose of 150 mg/kg during the first week and then at 200 mg/kg during 14 weeks to induce cirrhosis. Rats were stratified to treatment groups (n=5 per group) based on mean plasma levels of fibrosis markers (alpha-2 macroglobulin and Metallopeptidase Inhibitor 1 (TIMP1)) and liver function tests (plasma total bile acid and albumin) obtained from sublingual blood sampling.

Animals were caged in pairs on a 12:12-hour light-dark cycle, in environmentally controlled animal facilities, with ad libitum access to food and water. The body weight and the food intake were monitored twice a week.

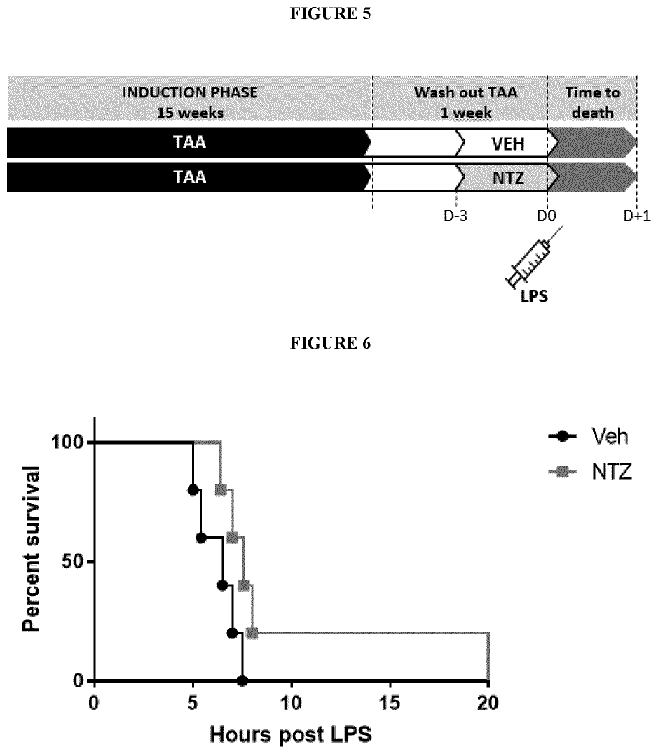

After cirrhosis induction, rats received a single intraperitoneal injection of 0.05 mg/kg lipopolysaccharide (LPS from Escherichia coli O111:64, Sigma-Aldrich) to induce ACLF, one week after TAA administration was stopped. Rats received NTZ by oral gavage at the dose of 50 mg/kg/day BID or vehicle for 3 days followed by a last dose 30 minutes before the induction of ACLF (FIG. 5). Plasma were obtained from sublingual blood sampling just before sacrifice.

All animal procedures were performed according to standard protocols and in accordance with the standard recommendations for the proper care and use of laboratory animals.

### Plasma Analyses

As described in EXAMPLE 3, the plasmatic concentration of alpha-2 macroglobulin was determined by ELISA using the Abcam kit (cat #ab157730), and that of albumin was measured using the Randox kit for Daytona plus automate (Randox, cat #AB 8301).

The concentration of total bile acids was measured using the appropriate Randox kit for Daytona Plus automate (Randox, cat #BI 3863). In the presence of Thio-NAD, the enzyme 3-alpha hydroxysteroid dehydrogenase (3-a HSD) converts bile acids to 3-keto steroids and Thio-NADH. The reaction is reversible and 3-a HSD could convert 3-keto steroids and Thio-NADH to bile acids and Thio-NAD. In the presence of excess NADH, the enzyme cycling occurs efficiently and the rate of formation of Thio-NADH is determined by measuring specific change of absorbance at 405 nm.

The plasma TIMP-1 levels were measured using a quantitative sandwich ELISA assay from R&D Systems (cat #RTM100). The sample values are then calculated from the standard curve.

### Results

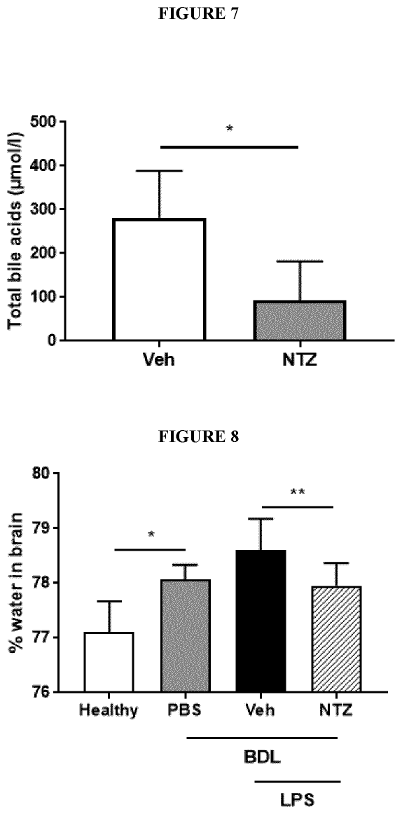

LPS administration in cirrhotic rats induced liver failure and mortality within 24 hours. In this experiment, NTZ treatment delayed the time to death of 1 hour and allowed the survival of 1 rat out of 5, whereas all the animals in the vehicle group died within 8 hours (FIG. 6). Also, NTZ reduced the circulating bile acids level suggesting improvement of liver functions (FIG. 7). Therefore, these results show that a short treatment of NTZ improves liver functions and protects from decompensation in a model of liver failure.

### Example 5: NTZ Improves Brain Edema in a Rodent Model of ACLF Induced by Bile Duct Ligation and LPS Injection

Acute-on-chronic liver failure is characterized by the deterioration of organs besides the acute decompensation of the liver. This study investigated the effect of NTZ on brain edema in a model of ACLF induced in rats by bile duct ligation (BDL)—induction of liver injury and advanced fibrosis in 3 weeks—and lipopolysaccharide (LPS) administration.

As described by Kountouras et al (British journal of Experimental Pathology 1984), rats that underwent BDL had severe liver injury with advanced fibrosis and bile duct proliferation 22 days post-surgery.

BDL surgery was performed on 37 Sprague Dawley rats weighing approximately 270 g at Charles River laboratory (France). After anesthesia with isoflurane (Vetflurane, Alcyon, France), and cutaneous application of 2% lidocaine (Alcyon), a median laparotomy was performed to expose the liver and duodenum. The main bile duct was identified and dissected. After that, the bile duct was ligated in two parts: a first ligation was made in the middle of the bile duct, and the second ligation was made above the entrance of the pancreatic duct. The bile duct was then cut in the middle, to avoid recanalization.

Animals were pretreated before surgery with 0.05 mg/kg of buprenorphine (Buprecare, Alcyon) and 5 mg/kg of Carprofen (Carprofelican, Alcyon) and received a dose of 0.05 mg/kg of buprenorphine 4 hours and then a dose of 5 mg/kg of Carprofen 24 and 48 hours after surgery.

The animals were then transferred to Genfit's animal facility (France) after a minimum of 5 days of recovery post-surgery. Fifteen days after surgery, a blood sample was collected from the sublingual vein and under light isoflurane (Isoflurin, Axience, France) anesthesia to measure markers of hepatic injury (serum aspartate aminotransferase (AST) and alkaline phosphatase (ALP)) to stratify the animals into treatment groups. Two BDL rats were excluded at this stage for abnormal blood parameters and 4 died or were killed for ethical reason (10-20% early mortality is expected with BDL surgery). Unoperated animals were included in the study as healthy controls.

Twenty-two days after BDL surgery, acute decompensation was induced by intraperitoneal administration of 1 μg/kg LPS (Escherichia coli O111:64, Sigma-Aldrich, Germany). Four rats with BDL received phosphate buffer saline (PBS, Fisher Scientific, USA) instead of LPS to be used as BDL controls. Each animal was continuously observed following LPS injection in order to assess the degree of severity of the inflammatory response and pain. LPS injection in these rats induced a high inflammatory response clearly visible with the change of ear color into red.

Treatment with NTZ 100 mg/kg (Interchim, France) or vehicle (1% carboxymethylcellulose (C4888, Sigma-Aldrich), 0.1% tween 80 (P8074, Sigma-Aldrich)) was administered by gavage just before LPS injection.

Three hours after LPS injection, blood sampling was performed from the sublingual vein and under light anesthesia as previously described, and the animals were euthanized by cervical dislocation. Liver, spleen, kidney and brain were collected.

Manipulation of animals was conducted carefully in order to reduce stress at the minimum. All the experiments were performed in compliance with the guidelines of French Ministry of Agriculture for experiments with laboratory animals (law 87-848). The study was conducted in compliance with Animal Health Regulation (Council directive No. 2010/63/UE of Sep. 22, 2010 and French decree no. 2013-118 of Feb. 1, 2013 on protection of animals).

| | | Animal/ | |
|---|---|---|---|
| Group | Compounds | group | Comment |
| Healthy | none | 4 | Unoperated healthy |
| | | | control |
| BDL + PBS | none | 4 | Liver fibrosis |
| | | | control terminated |
| | | | 22 days post-BDL |
| BDL + LPS + veh | none | 13 | ACLF control |
| | | | terminated 22 |
| | | | days post-BDL |
| | | | and 3 hours |
| | | | post-LPS |
| BDL + LPS + NTZ | NTZ | 13 | NTZ treatment |
| | (100 mg/kg) | | terminated 22 |
| | | | days post-BDL |
| | | | and 3 hours post-LPS |

To evaluate brain edema, fresh brains were weighted, dehydrated by heating for 4 hours, and weighed again to calculate the percentage of water.

### Results

Liver injury was associated with a significant increase in brain edema, as assessed by the % of water contained in brain (FIG. 8). NTZ administration concomitant to LPS injection remarkably reduced brain water. This experiment thus demonstrates that NTZ can be used to treat or prevent brain edema.

### Example 6: NTZ Improves Kidney Function in a Rodent Model of ACLF Induced by Bile Duct Ligation and LPS Injection

This study investigated the effect of NTZ on markers of kidney function in a model of ACLF induced in rats by bile duct ligation (BDL)—induction of liver injury and advanced fibrosis in 3 weeks—and lipopolysaccharide (LPS) administration.

### Material & Methods

BDL surgery of rats and acute decompensation were performed as in the previous example.

| | | Animal/ | |
|---|---|---|---|
| Group | Compounds | group | Comment |
| Healthy | none | 4 | Unoperated healthy |
| | | | control |
| BDL + PBS | none | 4 | Liver fibrosis |
| | | | control terminated |
| | | | 22 days post-BDL |
| BDL + LPS + veh | none | 13 | ACLF control |
| | | | terminated 22 |
| | | | days post-BDL |
| | | | and 3 hours |
| | | | post-LPS |
| BDL + LPS + NTZ | NTZ | 13 | NTZ treatment |
| | (100 mg/kg) | | terminated 22 |
| | | | days post-BDL |
| | | | and 3 hours |
| | | | post-LPS |

Renal injury was evaluated by serum cystatin C concentrations determined by ELISA (Mouse/Rat Cystatin C Immunoassay Quantikine® ELISA, MSCTC0, R&D Systems).

### Results

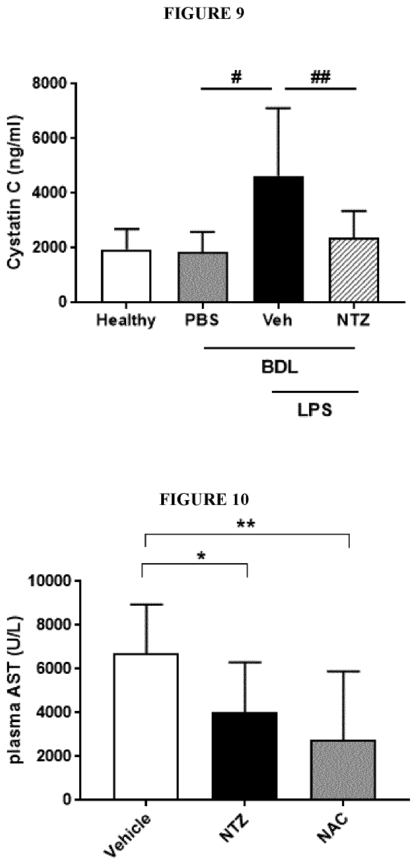

LPS injection induced renal alteration, as shown by increased serum cystatin C level (FIG. 9). NTZ treatment drastically reduced cystatin C, almost to the level of healthy controls. Taken together, these results demonstrate the potency of NTZ to improve peripheral organ (in particular brain, and kidney) injury in ACLF.

### Example 7: NTZ Reduces Liver Injury in Acetaminophen-Induced Acute Liver Failure

Acute liver failure was induced by administration of toxic dose of acetaminophen (APAP) in mice. After a 5-day acclimation period, 8 week-old C57BL/6J male mice (Janvier, France) were stratified into 3 groups of 10 mice according to their body weight. All mice were fasted overnight before the administration of 300 mg/kg APAP by intraperitoneal injection. Thirty minutes before intoxication with APAP, mice received intragastric gavage with vehicle (1% carboxymethylcellulose (C4888, Sigma-Aldrich), 0.1% tween 80 (P8074, Sigma-Aldrich)), 50 mg/kg NTZ (Interchim, France) or 1200 mg/kg N-acetylcysteine (NAC), the reference treatment for APAP intoxication. Mice had free access to food and water right after APAP injection.

Six hours after APAP injection, blood was collected in all mice on heparin by retro-orbital sinus punction under anesthesia. Liver injury was assessed by plasma aspartate aminotransferase (AST) determined using a Horiba Pentra 400 machine and related Pentra assay kit (Horiba France SAS, France). Animals were sacrificed 24 hours post-APAP administration by cervical dislocation, exsanguinated with saline and liver samples were collected and weighed.

Manipulation of animals was conducted carefully in order to reduce stress at the minimum. All the experiments were performed in compliance with the guidelines of French Ministry of Agriculture for experiments with laboratory animals (law 87-848). The study was conducted in compliance with Animal Health Regulation (Council directive No. 2010/63/UE of Sep. 22, 2010 and French decree no. 2013-118 of Feb. 1, 2013 on protection of animals).

### Results

The results show that APAP intoxication induced a dramatic increase in plasma AST (>6000 U/I compared to <100 U/I in healthy mice). Surprisingly, NTZ treatment greatly reduced AST to a level similar to that with NAC, the treatment of reference for APAP intoxication (FIG. 10). These results show the ability of NTZ to treat patients with drug-induced acute liver failure (ALF).

### Example 8: NTZ Improves Renal Function Markers

Cirrhosis decompensation often associates with alterations of kidney functions. This study investigated the effect of NTZ to prevent kidney injury in a model of acute-on-chronic liver failure (ACLF). Briefly, ACLF was induced by lipopolysaccharide (LPS) injection in rats with advanced liver fibrosis/cirrhosis induced by repeated carbon tetrachloride (CCl₄) administrations for 15 weeks.

The protocol study included 4 phases:
1) Pre-sensitization phase: phenobarbital (P5178, Sigma Aldrich) was added to the drinking water (35 g/dl) of 32 4-week-old male Sprague-Dawley rats (˜200 gr, Janvier Lab) for 2 weeks in order to activate cytochrome enzymes that metabolize CCl₄.
2) Induction of liver fibrosis: chronic liver damages were induced by repeated (twice a week) oral CCl₄ (ref: 289116, Sigma Aldrich; diluted in olive oil) administrations (increasing doses starting with 0.10 ml/kg, adding 0.05-0.10 ml/kg at each administration to reach 0.85 ml/kg at 7 weeks and thereafter) for up to 15 weeks, with final doses as follows:

| | |
|---|---|
| Week 1 | 0.10 ml/kg |
| | 0.20 ml/Kg |
| Week 2 | 0.25 ml/Kg |
| | 0.30 ml/Kg |
| Week 3 | 0.35 ml/Kg |
| | 0.40 ml/Kg |
| Week 4 | 0.45 ml/Kg |
| | 0.50 ml/Kg |
| Week 5 | 0.60 ml/Kg |
| | 0.70 ml/Kg |
| Week 6 | 0.80 ml/Kg |
| | 0.85 ml/Kg |
| Week 7 and | 0.85 ml/kg |
| thereafter | |

3) Treatment phase: after 15 weeks of CCl₄ administration, CCl₄ gavage was stopped one week before LPS administration. Vehicle (1% carboxymethylcellulose (C4888, Sigma-Aldrich), 0.1% tween 80 (P8074, Sigma-Aldrich)) or NTZ (50 mg/kg BID) were administered orally during 3 days before LPS challenge. A last dose (50 mg/kg) of either vehicle or NTZ was administered 30 minutes just before LPS challenge.
4) LPS challenge: LPS from Escherichia coli O111:64 (L2630, Sigma Aldrich) was intraperitoneally injected at 0.03 mg/kg.

| Group | Compounds | Animal/group | Comment |
|---|---|---|---|
| Olive oil | none | 6 | Non pathological |
| W10 | | | control, terminated |
| | | | at Week 10 |
| CCl₄ W10 | none | 12 | Liver fibrosis control, |
| | | | terminated at Week 10 |
| ACLF-Veh | none | 10 | LPS challenge after |
| | | | 15 weeks CCl₄ |
| ACLF-NTZ | NTZ | 10 | LPS challenge after |
| | (50 mg/kg BID) | | 15 weeks CCl₄ |

During the first hours following LPS administration, each animal was continuously observed in order to assess the degree of severity of the inflammatory response and pain. The appearance of the animal (change in fur, skin color), the degree of activity and vigilance (response to a stimulus, opening of the eyelids, activity . . . ), gait and attitude were monitored. Any animal showing evident signs of suffering and close to death was euthanized. In both NTZ and vehicle groups, 6 animals out of 10 survived the LPS injection. All surviving animals were euthanized 24 h after LPS challenge for plasma and tissue analyses. All animal procedures were performed according to standard protocols and in accordance with the standard recommendations for the proper care and use of laboratory animals.

Renal injury was evaluated by plasma creatinine, urea and cystatin C concentrations. Plasma creatinine and urea concentrations were measured using the appropriate Randox kits (CR 8316, UR 8334, respectively) for Daytona plus automate (Randox, cat #BR 8377) using the manufacturer instructions. Plasma cystatin C concentration was determined by ELISA (Mouse/Rat Cystatin C Immunoassay Quantikine® ELISA, MSCTC0, R&D Systems).

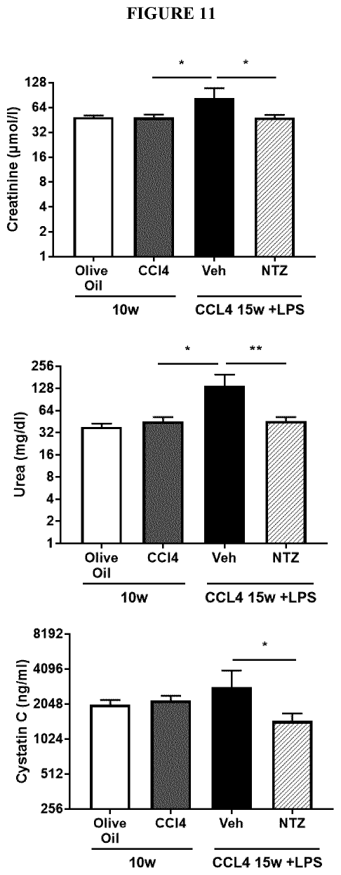

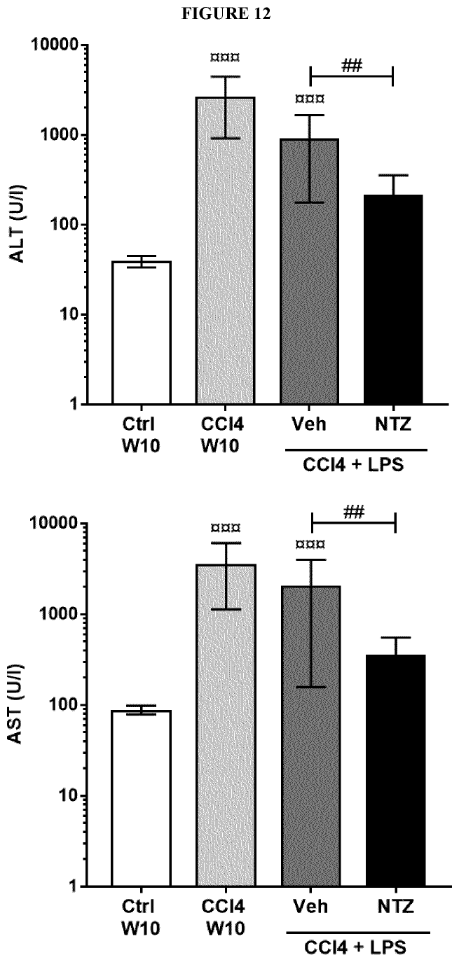

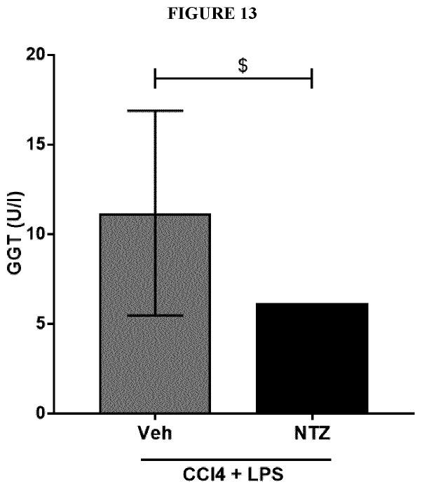

The results show that LPS injection after 15 weeks of CCl4 administrations worsens kidney functions, as shown by increase in plasma creatinine, urea and cystatin C concentrations (FIG. 11). NTZ treatment remarkably prevents kidney injury by restoring creatinine, urea and cystatin C levels to the levels of the olive oil controls.

### Example 9: Evaluation of NTZ Efficacy on CCl4+LPS-Induced Acute-On-Chronic Liver Failure in Rats

The aim of the study was to assess the efficacy of NTZ to prevent LPS-induced hepatic and renal injuries in Sprague-Dawley rats with CCl₄-induced severe fibrosis.

This study included 4 phases:
1) Pre-sensitization phase: phenobarbital (35 g/dL) (ref P5178-25G, Sigma-Aldrich) was added to the drinking water of 4-week-old male Sprague-Dawley rats (˜200 g) for 2 weeks prior to CCl₄ administration
2) Induction phase: hepatic severe fibrosis was induced by repeated oral tetrachlorure (CCl₄) administrations (twice a week, increasing doses from 0.10 to 0.85 ml/kg, twice weekly) for 10 or 15 weeks, with the same protocol as the protocol described in example 8.
3) Treatment phase: after 15 weeks of CCl₄ administrations, gavage was stopped one week before LPS administration. Vehicle or NTZ (50 mg/kg bis in die (BID)) was administered orally during 3 days before ACLF induction. A last dose (50 mg/kg) was administered 30 minutes just before ACLF induction.
4) ACLF induction: LPS was administrated i.p. at 0.03 mg/kg.

For oral gavage, NTZ powder (ref RQ550, Interchim) was dissolved in CMC 1% in water (sodium Carboxymethyl cellulose ref C48 88-500G, Sigma Aldrich), Tween 80 0.1% in water at the concentration of 10 mg/mL in an amber glass bottle, homogenized with a polytron and sonicated 10 seconds at a power of 10%. NTZ was kept under magnetic stirring until the administration to the rats at 10 mL/kg and protected from light.

LPS solution (Escherichia coli O111:64, Sigma-Aldrich, Germany) was prepared under a microbiological safety cabinet and was dissolved in phosphate buffer saline (PBS) at 15 μg/mL, aliquoted and frozen until the day of experiment. LPS was administered at 2 m L/kg to the rats.

38 male Sprague-Dawley rats (Janvier Labs, France) have been allocated to this study. Animals had ad libitum access to water and chow diet (ref E15000-04, Ssniff, Germany) all along the study. After 9 weeks of CCl₄ administration, animals were randomly allocated to 2 groups according to ammonia and albumin plasmatic levels (in order to have similar means in the two groups).

All surviving animals were euthanized 24 h after LPS injection for plasma and tissues analyses (60% survived in both vehicle and NTZ groups). ACLF was evaluated, at the end of the study, by biochemical analyses of hepatic and renal injury or function biomarkers.

All animal procedures were performed according to standard protocols and in accordance with the standard recommendations for the proper care and use of laboratory animals according to European Directive 2010/63UE.

The body weight and food intake were monitored twice a week all along the study.

During the fibrosis induction phase, each animal was observed once a day for the monitoring of clinical signs. Observations include changes in the skin, fur, eyes, occurrence of secretions and excretions and autonomic activity (lachrymation, piloerection, unusual respiratory pattern). Changes in gait, posture, stereotypes (e.g. excessive grooming, repetitive circling) or bizarre behavior (self-mutilation, walking backwards . . . ) are also monitored. Animals were euthanized in case of weight loss of more than 25% for more than 3 consecutive days, absence of food consumption, deterioration of the general condition or vocalization.

After LPS administration, each animal was continuously observed during 7 hours in order to assess the degree of severity of the inflammatory response and pain. A scoring table taking into account the appearance of the animal (change in fur, skin color), the degree of activity and vigilance (response to a stimulus, opening of the eyelids, activity . . . ), gait and attitude were assessed. Animals were euthanized in case of deterioration of the general condition of the animal (cold to the touch, unable to rise, vocalization, dyspnea).

At the end of the study, blood was collected by sublingual vein puncture under light isoflurane anesthesia and transferred to ethylenediamine tetra-acetic acid (EDTA), serum-gel, and lithium heparin tubes. EDTA and lithium heparin tubes containing blood were rapidly centrifuged and the plasma fraction was collected. Plasma aliquot of heparin lithium were snap frozen in liquid nitrogen and stored at −80° C., EDTA-plasma aliquots were stored at −20° C. and serum-gel tubes were kept at room temperature for 30 minutes, centrifuged and stored at −20° C.

Then, animals were euthanized by cervical dislocation, and beheaded for brain excision and weighing. The liver, spleen and right kidney were collected and weighed. A liver slice was fixed and embedded in paraffin for histological analyses. The remaining liver was snap frozen in liquid nitrogen and kept at −80° C. for biochemical analyses.

### Histological Sample Processing and Analyses:

The liver slices were first fixed for 10 hours or 24 hours (week-end protocol) in formalin 4% solution (Merck Sigma). Then, the liver pieces were dehydrated in ethanol solutions (baths at 70 and 100% ethanol). The liver pieces were incubated in two baths of Isopropanol, followed by two baths in liquid paraffin (60° C.). These steps were performed on LOGOS One (Milestone, MicromMicrotech). Liver pieces were then put into racks (Fisher scientific, ref 11670990) that were gently filled with Histowax® (Microm, ref F/00403) to completely cover the tissue. The paraffin blocks containing the tissue pieces were removed from the racks and stored at room temperature. The liver blocks were cut into 3 μm slices on Electronic Rotary Microtome HM340E (ThermoScientific, MicromMicrotech).

Picrosirius Red Fast Green staining was performed on the Autostainer ST5030 (Leica). Liver sections were deparaffinized, rehydrated and incubated for 15 minutes in Fast Green 0.04% solution (Sigma, cat #F7258-25G). Then, the liver sections were rinsed in acetic acid 0.5% solution and incubated 30 minutes in Picrosirius Red 0.1% (Direct red, Alfa Aesar, cat #621693-25G and Picric acid solution, Sigma, cat #P6744)—Fast Green 0.04% solution. Sections were dehydrated and mounted using the CV Mount medium (Leica, cat #14046430011) on the automated coverslip CV5030 (Leica). The histological examinations and scoring were performed blindly to the treatment group. Images were acquired using Pannoramic 250 Flash II digital slide scanner (3DHistech) and analyzed in QuantCenter software.

Of note, only the animals (60%) that survived 24 h post-LPS could be analyzed.

Experimental results are expressed as mean±standard deviation and plotted as bar graphs. Comparison between groups was tested using a Student T test for all variables that follows a normal distribution (#: p<0.05; ##: p<0.01; ###: p<0.001). A Welsh test was applied in case of different variances between groups (3a: p<0.05; cm: p<0.01; man: p<0.001). A log-transformation was applied for plasma ALT and AST in all samples to obtain a normal data distribution. For other non-normally distributed variables, a non-parametric Mann-Whitney test was applied ($: p<0.05; $$: p<0.01; $$$: p<0.001). Differences to the Ctrl week 10 (W10) group are notified, if not otherwise specified.

### Results

Repeated CCl₄ administrations induced a severe liver fibrosis characterized by F3 score in most animals.

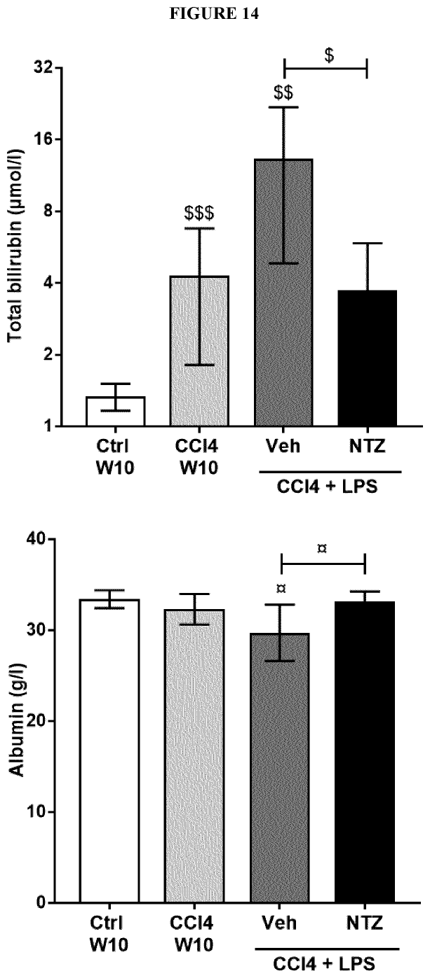

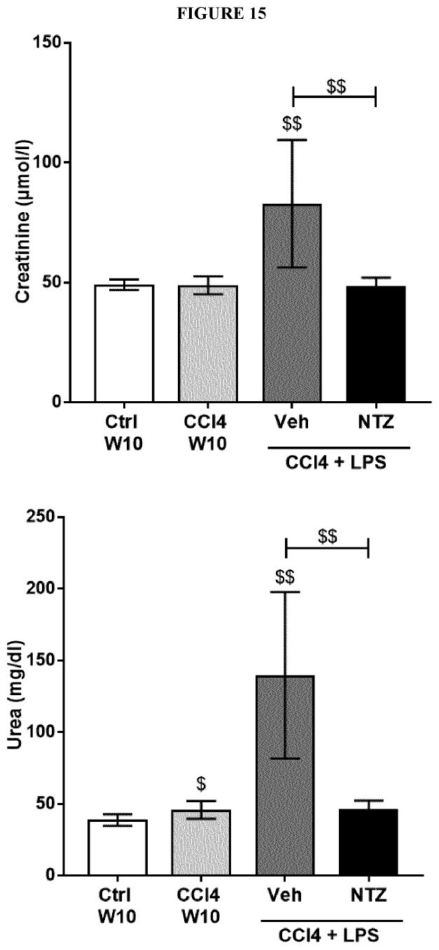

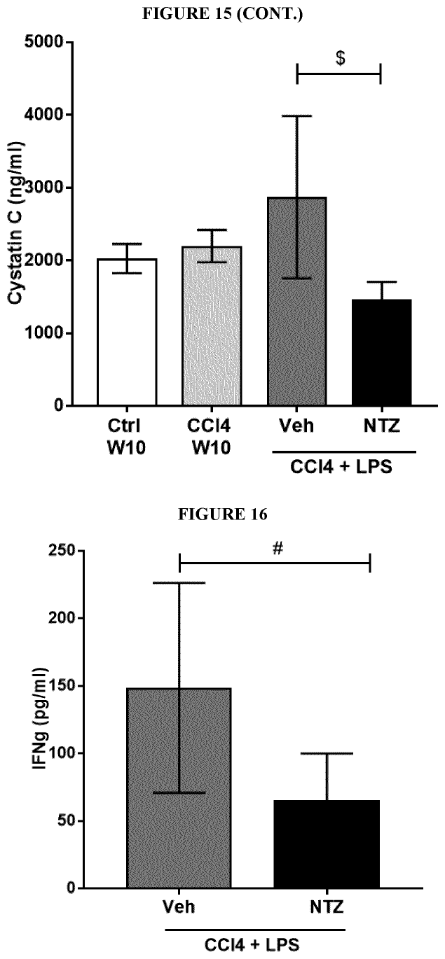

ACLF induction by LPS led to a significant mortality over 24 hours of 40% in both vehicle and NTZ-treated groups (4/10 rats in both groups). Hepatic and renal injury and function markers were evaluated in the surviving animals 24 hours post-LPS. Plasma ALT and AST levels, markers of hepatocellular injury, were highly increased by CCl₄ whereas NTZ treatment greatly reduced ALT by 80% (p=0.007) and AST by 87% (p=0.005) (FIG. 14). Plasma GGT, another marker of hepatic damage, was not altered by CCl₄ administrations (not detected) while LPS injection increased plasma GGT level to 11.2 U/I (FIG. 15). NTZ treatment blunted this LPS-induced rise in GGT (<6.15 U/I).

Plasma total bilirubin and albumin—markers of hepatic functions—were also altered with CCl₄ and LPS administrations (FIG. 16). NTZ treatment prevented LPS-induced alteration of hepatic function as shown by 80% reduction of total bilirubin (p=0.02) and restoration of albumin production by 95% (p=0.04).

LPS injection also altered renal functions as shown by elevated plasma creatinine, urea and cystatin C concentrations (FIG. 17), confirming that organs other than the liver are affected in this ACLF model. NTZ treatment also prevented LPS-induced renal impairment by reducing plasma creatinine (−102%, p=0.009), urea (−92%, p=0.002) and cystatin C (−166%, p=0.02).

Serum IFNγ was measured in the animals that received CCl₄ for 15 weeks and LPS, while other cytokines (IL6, TNFα, IL1β) could not be detected (not shown). NTZ treatment reduced significantly serum IFNγ by 56% (p=0.04) which confirms its anti-inflammatory effects in an ACLF model (FIG. 18).

### CONCLUSION

In rats with severe fibrosis (F3/F4), a 3-day pretreatment with NTZ 100 mg/kg/day alleviates hepatic damages (ALT, AST, GGT) and prevents LPS-induced alterations of hepatic (total bilirubin, albumin) and renal (creatinine, urea, cystatin C) functions.

1. A method for treating or preventing a liver failure in a subject in need thereof, the method comprising administering a compound to the subject, wherein the compound is selected from the group consisting of nitazoxanide (NTZ), tizoxanide (TZ), tizoxanide glucuronide (TZG), and pharmaceutically acceptable salts thereof, and wherein the liver failure is acute on chronic liver failure (ACLF) or acute liver failure (ALF).

2-3. (canceled)

4. The method of claim 1, for preventing the progression from compensated cirrhosis to decompensated cirrhosis in a subject having ACLF.

5. The method of claim 1, wherein the subject has ACLF and compensated cirrhosis.

6. (canceled)

7. The method of claim 1, for reversing decompensated cirrhosis to compensated cirrhosis in a subject having ACLF.

8. The method of claim 1, wherein the subject has ACLF grade 2 or 3.

9. The method of claim 1, wherein the subject has at least one ACLF precipitating event.

10. (canceled)

11. The method of claim 1, wherein the subject has ACLF provoked by a hepatic precipitating condition or by an extrahepatic precipitating condition.

12. The method of claim 1, for preventing liver decompensation in a subject having ACLF.

13. (canceled)

14. The method of claim 1, wherein the ALF is drug-induced ALF.

15. The method of claim 14, wherein the drug-induced ALF is acetaminophen-induced ALF.

16. A method of preventing extrahepatic organ failure in a subject having cirrhosis, the method comprising administering a compound to the subject, wherein the compound is selected from the group consisting of nitazoxanide (NTZ), TZ(G), and pharmaceutically acceptable salts thereof.

17. The method of claim 16, wherein the subject has compensated or decompensated cirrhosis.

18. The method of claim 16, wherein the subject has decompensated cirrhosis or alcoholic cirrhosis.

19. (canceled)

20. The method of claim 16, for preventing kidney failure.

21. The method of claim 16, wherein the subject has ACLF without kidney failure.

22. The method of claim 16, wherein the subject has ACLF with a non-kidney organ failure and with kidney dysfunction.

23. The method of claim 1, for further preventing hepatic encephalopathy.

24. The method of claim 1, for further treating or preventing brain edema.

25. (canceled)

26. The method of claim 1, the method further comprising administering a liposomal-based toxin scavenger to the subject.

27. The method of claim 26, wherein the liposomal-based toxin scavenger is an ammonium scavenger.

28. (canceled)
